(19) United States
(12) Patent Application Publication
(10) Pub. No.: US 2017/0347940 A1
(43) Pub. Date: Dec. 7, 2017

(54) SMART BANDAGE

(71) Applicant: Occammd, LLC, Tampa, FL (US)

(72) Inventor: Raymond A. Carr, Lutz, FL (US)

(21) Appl. No.: 15/615,389

(22) Filed: Jun. 6, 2017

Related U.S. Application Data
(60) Provisional application No. 62/346,075, filed on Jun. 6, 2016.

(51) Int. Cl. A61B 5/00; A61B 5/01; A61M 1/00; G08B 21/04; H04B 5/02; A61B 5/145; H04M 1/725; H04B 5/00

(52) U.S. Cl. CPC A61B 5/445; H04M 1/7253; A61B 5/01; H04B 5/0075; H04B 5/0037; G08B 21/0453; H04B 5/02; A61B 5/145; A61B 5/14539; A61B 5/4842; A61M 1/0084; A61M 2205/3303; A61M 2205/18; A61M 2205/3344; A61M 2205/3592; A61M 2205/50; A61M 2205/52; A61M 2205/8206

(57) ABSTRACT

The disclosure describes a smart bandage assembly. The bandage assembly includes a bandage member; a movement sensor, a pressure sensor, a methane sensor, or a humidity sensor; a microcontroller; a memory; and a radio transceiver for communication with a mobile communications device. The electronic components of the bandage assembly may be embedded within the bandage member. In some implementations, the radio transceiver harvests RF energy to power the memory within the bandage assembly so that sensor data stored in the memory can be accessed by a mobile communications device when other remaining electronic devices in the bandage assembly remain in a sleep state or power off mode.

### CROSS REFERENCE TO RELATED APPLICATIONS

This application claims the benefit of U.S. Patent Application No. 62/346,075 filed Jun. 6, 2016. The disclosure of which is hereby incorporated herein by reference in its entirety.

### FIELD OF THE DISCLOSURE

The present disclosure relates to medical devices and more particularly to the monitoring of a person's health and assistance with treatment of wounds through use of a smart bandage or patch.

### BACKGROUND

Smart bandages or bandages that include some electronic components together with a medication dispensing mechanism have been recently developed. For example, a paper published in “Advanced Materials” (http://web.mit.edu/zhaox/www/pappers/79.pdf) discloses a bandage made with a flexible material and having a temperature sensor. However, such bandage lacks complexity in terms of its wound monitoring and reporting capabilities. Therefore, there is a need in the art for a smart bandage that allows for improved monitoring and reporting capabilities. Further, smart bandages known in the prior art do not have the capacity of being commissioned or provisioned, and simply transmit data and in response to a “wake up” or “resend data” command from a remote device. Therefore, there is a need in the art for a smart bandage that can be commissioned or provisioned in the field and that includes circuitry that allows a greater level of interaction with a remote device or reader.

### SUMMARY

The following presents a simplified summary of the disclosure in order to provide a basic understanding of some aspects of the disclosure. This summary is not an extensive overview of the disclosure. It is intended to neither identify key or critical elements of the disclosure nor delineate the scope of the system and method disclosed herein. Its sole purpose is to present some concepts of the disclosure in a simplified form as a prelude to the more detailed description that is presented later.

In accordance with one implementation, the present disclosure describes a smart bandage that includes a movement sensor, a pressure sensor, a methane sensor, a humidity sensor and/or a thermometer that may be embedded in the bandage. In one implementation, a microcontroller with associated RAM memory, an NFC transceiver, a battery, and an EEPROM, which may be mounted, integrated or embedded in the bandage. In one implementation, the smart bandage may be in communication with a multi-function mobile device such as a smart phone or tablet running an application that displays data obtained from the bandage and that creates alerts to be displayed to a person wearing the bandage, for example.

The following description and the annexed drawings set forth in detail certain illustrative aspects of the disclosure. These aspects are indicative; however, of but a few of the various ways in which the principles of the system and method disclosed herein may be employed. The system and method disclosed herein is intended to include all such aspects and their equivalents. Other advantages and novel features of the system and method disclosed herein will become apparent from the following detailed description of the system and method disclosed herein when considered in conjunction with the drawings.

By commissioning the bandage on the fly, for example, use of the bandage disclosed herein will result in a fast exchange of sensor readings on the field which will allow the creation of medical charts.

### BRIEF DESCRIPTION OF THE DRAWINGS

The accompanying drawings, which are incorporated in and constitute a part of this specification, show certain aspects of the present disclosure and, together with the description, help explain some of the principles associated with the disclosure.

FIG. 1 illustrates a smart bandage with integrated sensors in accordance with one implementation of the present disclosure.

FIG. 2 illustrates a smart bandage with additional integrated components in accordance with one implementation of the present disclosure.

FIG. 3 illustrates different sensors which may be used with the smart bandage in accordance with one implementation of the present disclosure.

### ILLUSTRATIVE IMPLEMENTATIONS OF THE DISCLOSURE

Reference will now be made in detail to implementations, examples of which are illustrated in the accompanying drawings. The following description refers to the accompanying drawings, in which, in the absence of a contrary representation, the same numbers in different drawings represent similar elements. The implementations set forth in the following description do not represent all implementations consistent with the claimed invention. Instead, they are merely some examples of systems and methods consistent with certain aspects related to the disclosure. These implementations, which are also referred to herein as “examples,” are described in sufficient detail to enable those skilled in the art to practice the subject matter disclosed herein. It is to be understood that the implementations may be combined or that other implementations may be utilized, and that structural, logical, and electrical variations may be made without departing from the scope of the subject matter disclosed herein. The following detailed description is, therefore, not to be taken in a limiting sense.

As used herein, an element or step recited in the singular and preceded with the word “a” or “an” should be understood as not excluding plural of said elements or steps, unless such exclusion is explicitly stated. In this document, the term “or” is used to refer to a nonexclusive or, unless otherwise indicated. Furthermore, references to “one implementation” are not intended to be interpreted as excluding the existence of additional implementations that also incorporate the recited features. Moreover, unless explicitly stated to the contrary, implementations “comprising” or “having” an element or a plurality of elements having a particular property may include additional such elements not having that property.

Implementations of applications executed by portable multifunction devices, user interfaces for such devices, and associated processes for using such devices are described. In some implementations, the device is a portable communications device such as a mobile telephone that also contains other functions, such as PDA and/or music player functions. The device may also be a tablet, smart phone, or the like.

As used herein, the term “computer” or “module” may include any processor-based or microprocessor-based system including systems using microcontrollers, reduced instruction set computers (RISC), application specific integrated circuits (ASICs), field-programmable gate arrays (FPGAs), graphical processing units (GPUs), logic circuits, and any other circuit or processor capable of executing the functions described herein. The above examples are exemplary only, and are thus not intended to limit in any way the definition and/or meaning of the term “computer.”

The computer or processor executes a set of instructions that may be stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various implementations of the disclosure. The set of instructions may be in the form of a software program, which may form part of a tangible non-transitory computer readable medium or media. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to operator commands, or in response to results of previous processing, or in response to a request made by another processing machine.

As used herein, the terms “software”, “firmware” and “algorithm” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

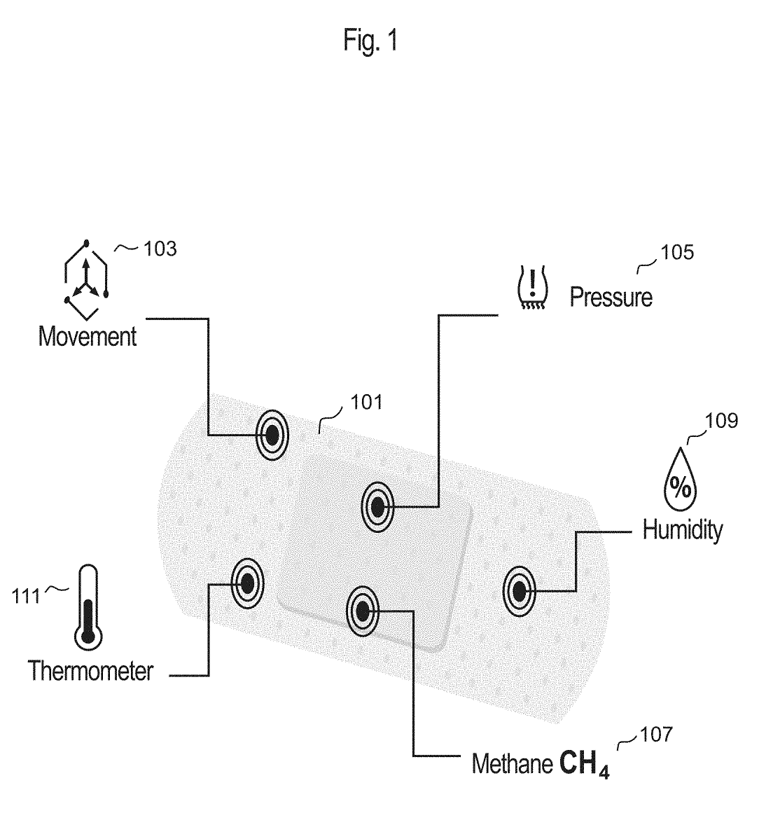

FIG. 1 illustrates a smart bandage 101 with integrated sensors in accordance with one implementation of the present disclosure. FIG. 1 illustrates a smart bandage that includes a movement sensor 103, a pressure sensor 105, a methane sensor 107, a humidity sensor 109 and a thermometer 111 that may be embedded in the bandage.

The movement sensor 103 may be implemented as an accelerometer which may be used to ensure a wound is healing by notifying a patient if he or she is moving too frequently or too much. The methane sensor 107 may be defined as a chemical sensor, especially monitoring methane, which may be used to detect tissue death before it can be visually detected.

The pressure sensor 105 may be used to monitor edema, which is typically present in diabetic or pressure ulcers, for example. The humidity sensor 109 may be used to monitor whether a wound has exudate or not, and how the presence of exudate correlates to the healing process. The thermometer 111 or temperature sensor may be used to ensure that a wound is kept warm, with the goal of ultimately advising the patient or caretaker to keep the wound at a certain temperature (e.g., close to 98.6° F.).

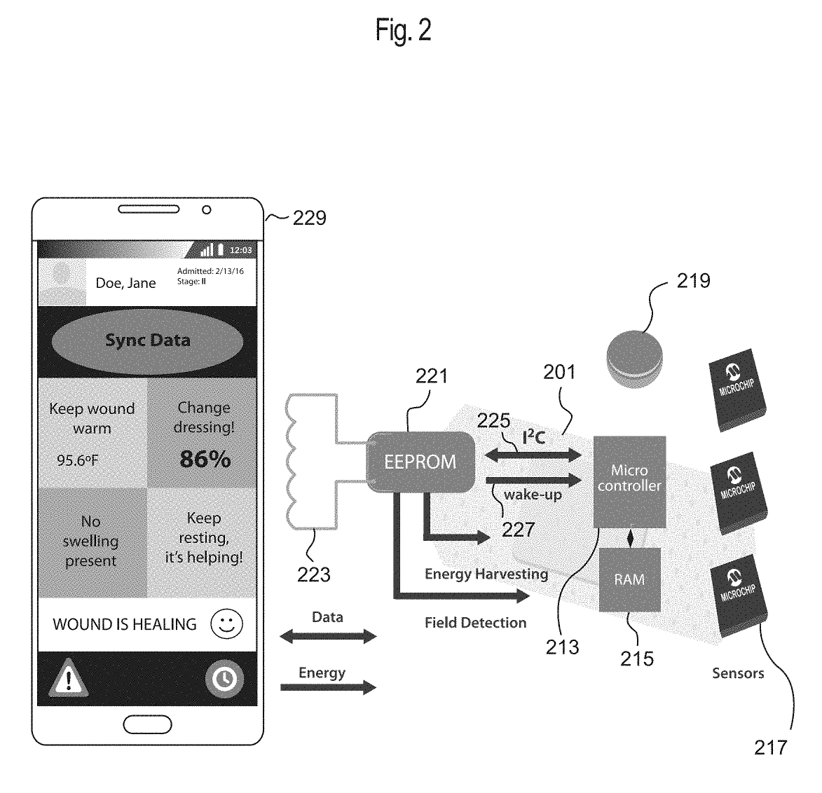

FIG. 2 illustrates a smart bandage 201 with additional integrated components in accordance with one implementation of the present disclosure. FIG. 2 illustrates a microcontroller 213 with an associated RAM 215, several sensors 217, and a “button” battery 219 used to provide power to the sensors 217 and the microcontroller 213. FIG. 2 also illustrates an EEPROM 221 and an NFC transceiver 223 represented by the induction coil in the figure. The NFC transceiver 223 may include the AS3953 NFC Interface Tag available from ams (http://ams.com/eng).

The EEPROM 221 may be used to store data collected by the sensors 217, with the microcontroller controlling or directing the operation of the sensors 217 and the transfer of data from the sensors to the EEPROM 221 through use of an I-squared-C bus 225, for example. In one implementation, a wakeup signal 227 is generated by the interaction of the NFC transceiver on the multi-function device 229 with the NFC transceiver 223 on the bandage when the two are within a field of detection. For example, the NFC transceiver on the multi-function device may generate the power required to read/write/wakeup the smart bandage 201. In one implementation, an NFC transceiver 223 in the bandage is connected to the EEPROM 221. The NFC transceiver 223 in the bandage can harvest energy from RF energy received from the multi-function mobile device's NFC transceiver through the transceiver (223) antenna's inductive coupling. The NFC transceiver 223 in the bandage can operate on the harvested energy or alternatively in real-time by generating energy from the received RF power and thus the NFC transceiver 223 in the smart bandage requires no external power source in one implementation. The NFC transceiver 223 in the bandage can be used to provide power to the EEPROM 221 so that the NFC transceiver 223 in the bandage can obtain the data stored in the EEPROM 221 without being required to communicate with the remaining devices embedded in the bandage (microprocessor 213, sensors 217, RAM 215, etc.). An application running in the multi-function mobile device 229 (smart phone, tablet, etc.) may then access the monitored data through its NFC transceiver without having to communicate with the microcontroller 213 or sensors 217 directly. In one alternative implementation, the EEPROM 221 and the NFC transceiver 223 in the smart bandage may be powered by a battery 219 embedded in the bandage and the sensor or meter readings may be accessed in real-time.

In FIG. 2, two arrows drawn from the EEPROM 221 chip represent energy harvesting and field detection. The harvested energy, in addition to being used to power the NFC 223 and EEPROM 221, may also be used to power non-volatile memory, and other small circuitry. The “field detection” arrow represents a wake up signal sent to the other devices. In one implementation, the field detection arrow represents an “NFC field present” indicator. Upon receipt of the wake up signal, the microcontroller may then instruct the devices embedded within the bandage to operate in full power mode.

The NFC transceiver may be used to wake up the electronic devices within the bandage and to commission or provision those electronic devices. NFC allows a design variant/close physical requirement (e.g., inches) for wireless data communications, so it can be patient specific in a large group of patients if the bandage(s) is or are used in a hospital. It also can power the memory (provisioning, crypto information, status, etc.) and its limited processing power for the bandage allows certain operations without requiring power to be drawn from the main battery so that the battery is not connected to the primary circuitry until requested via the NFC interface. This allows reading and writing of some data (communication with memory and/or the processor) without utilizing the primary battery until the unit is actually ready for full use. Thus, use of the NFC transceivers makes it easier to provision/commission the bandage circuitry. Alternatively, a barcode/QPC on the bandage may be used to provision the system onto a network.

As part of the commissioning or provisioning process, all or some of the following actions will take place: exchange of encryption keys to enable encryption, enable the radio transceiver (e.g., WiFi access, Bluetooth pairing, etc.), and create a patient profile. Alternative provisioning actions may also take place as would be recognized by a person of skill in the art. For example, one can commission the circuitry (key exchange, version/type info, etc.) without going to full power, so that the main battery can still be disconnected. This may be accomplished when the NFC transceiver can harvest energy from RF energy received by the NFC. Thus, one of the advantages provided by the present disclosure is that certain information can be exchanged with the multi-function portable device through use of power from the NFC without actually using up the main battery. After commissioning, the sensor data may then be read with the mobile communications device. In an alternative implementation, the sensors can send raw data to the mobile communications device so that the temperature, methane concentration, etc., can be calculated by the mobile device.

In one implementation, the smart bandage 201 may be in communication with a multi-function mobile device 229 such as a smart phone or tablet running an application that displays data obtained from the bandage, analyzes the data and displays wound treatment instructions or alerts to a person wearing the bandage, for example.

In one implementation, after the radio transceiver in the bandage is ready for transmitting sensor data, it may be configured to operate in a sleep/watchdog mode. That is, the multi-function mobile device would only need to talk to the bandage every so often (heartbeat) or it will go back into a battery saving mode that may still sample crucial parameters and collect data, by allowing powering down non-necessary parts of the circuit. The main circuitry in the bandage may be awakened into full mode by the NFC transceivers again, or the multi-function mobile device may listen every so often.

In one scenario, when a patient is first applied the bandage, a first tablet or phone may wake up and commission the bandage electronics through an NFC “bump.” After the oximeter is read by the phone or tablet, the radio communications (WiFi for example) may go into sleep mode. A second phone or tablet may “bump” with the bandage and readily access the patient's ID by looking at the picture of the patient displayed in the phone or tablet. In one implementation, the memory in the bandage may store a history of readings which may be accessed by the phone or tablet.

Once the main battery starts to allow operation of the bandage electronics in full power, the sensors may start to collect data and transmit wirelessly to the multi-function mobile device through a WiFi connection, for example. In one implementation, after the radio transceiver is ready for transmitting sensor data, it may be configured to operate in a sleep/watchdog mode. That is, the multi-function mobile device would only need to talk to the bandage every so often (heartbeat) or it will go back into a battery saving mode that may still sample crucial parameters and collect data, by allowing powering down non-necessary parts of the circuit. The main circuitry may be awakened into full mode by the NFC transceivers again, or the multi-function mobile device may listen every so often. In either configuration, the sensor data may be read by the multi-function mobile device.

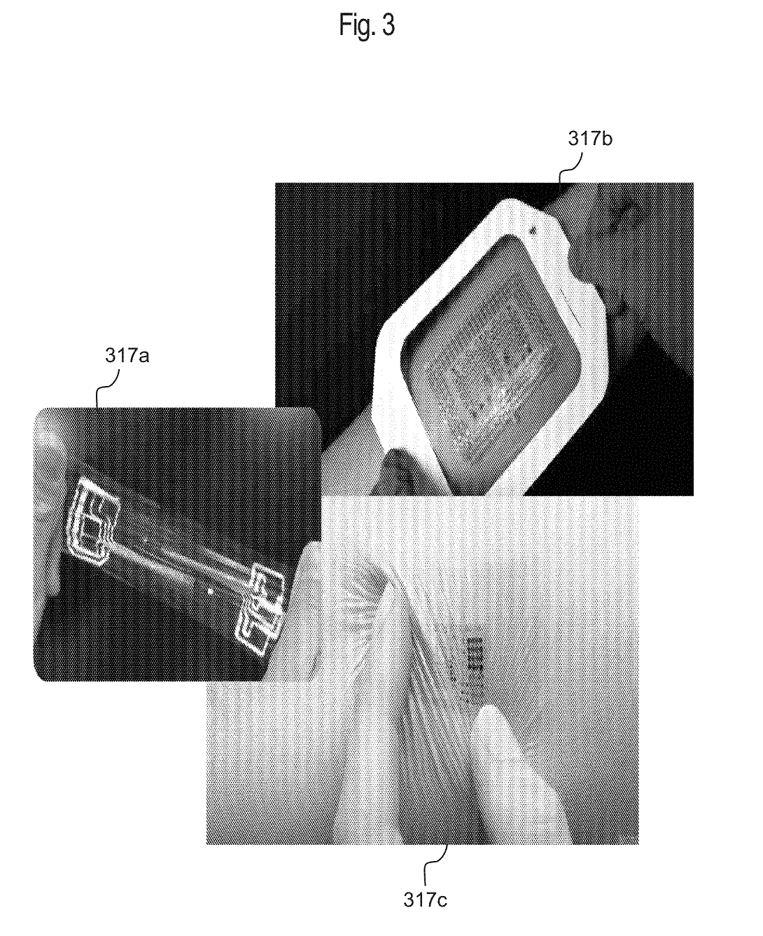

FIG. 3 illustrates different sensors 317a-c which may be used with the smart bandage in accordance with one implementation of the present disclosure. The illustrated sensors may be referred to as bio-stickers. These bio-sticker sensors may be embedded within the bandage 201 or attached thereto, for example. Other bio-sticker sensors may be used, as recognized by persons of skill in the art, for example, sensors described in the following link: http://www.slashgear.com/this-ultra-thin-electronic-skin-puts-a-digital-display-on-your-body-16436523/.

It is to be understood that the above description is intended to be illustrative, and not restrictive. For example, the above-described implementations (and/or aspects thereof) may be used in combination with each other. In addition, many modifications may be made to adapt a particular situation or material to the teachings of the disclosure without departing from its scope. Many other implementations will be apparent to those of skill in the art upon reviewing the above description. The scope of the disclosure should, therefore, be determined with reference to the appended claims, along with the full scope of equivalents to which such claims are entitled. In the appended claims, the terms “including” and “in which” are used as the plain English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112(F), unless and until such claim limitations expressly use the phrase “means for” followed by a statement of function void of further structure.

This written description uses examples to disclose the various implementations of the disclosure, including the best mode, and also to enable any person skilled in the art to practice the various implementations of the disclosure, including making and using any devices or systems and performing any incorporated methods. The patentable scope of the various implementations of the disclosure is defined by the claims, and may include other examples that occur to those skilled in the art. Such other examples are intended to be within the scope of the claims if the examples have structural elements that do not differ from the literal language of the claims, or if the examples include equivalent structural elements with insubstantial differences from the literal languages of the claims.

1. A smart bandage assembly comprising:
a bandage member;
a movement sensor, a pressure sensor, a methane sensor, or a humidity sensor;
a microcontroller;
a memory;
and
a radio transceiver for communication with a mobile communications device so that it is automatically commissioned by the mobile communications device when said radio transceiver is within a detection range of said mobile communications device.

2. The bandage assembly of claim 1, further comprising a thermometer.

3. The bandage assembly of claim 1, further comprising a medication dispensing mechanism.

4. The bandage assembly of claim 1, wherein the movement sensor includes an accelerometer that produces a signal that is input to the microcontroller or transmitted to the mobile communications device through the radio transceiver, said signal being processed by the microcontroller or the mobile communications device to generate an alert signal indicative of movement exceeding a predetermined frequency.

5. The bandage assembly of claim 1, wherein the methane sensor produces a signal that is input to the microcontroller or transmitted to a mobile communications device through the radio transceiver, said signal being processed by the microcontroller or the mobile communications device to generate an alert signal indicative of tissue death.

6. The bandage assembly of claim 1, wherein the pressure sensor produces a signal that is input to the microcontroller or transmitted to a mobile communications device through the radio transceiver, said signal being processed by the microcontroller or the mobile communications device to generate an alert signal indicative of the presence of edema.

7. The bandage assembly of claim 1, wherein the humidity sensor produces a signal that is input to the microcontroller or transmitted to a mobile communications device through the radio transceiver, said signal being processed by the microcontroller or the mobile communications device to generate an alert signal indicative of the presence of exudate.

8. The bandage assembly of claim 2, wherein the thermometer produces a signal that is input to the microcontroller or transmitted to the mobile communications device through the radio transceiver, said signal being processed by the microcontroller or the mobile communications device to generate an alert signal indicative of a temperature that falls below a predetermined threshold temperature.

9. The bandage assembly of claim 2, wherein said radio transceiver includes NFC circuitry which harvests radio frequency energy and supplies power, based at least on the harvested radio frequency energy, to the memory.

10. The bandage assembly of claim 2, wherein said memory comprises an EEPROM.

11. The bandage assembly of claim 2, further comprising a battery for supplying power to said microcontroller and to any of the movement sensor, the pressure sensor, the methane sensor, the thermometer, or the humidity sensor.

12. The bandage assembly of claim 10, wherein the EEPROM stores data collected by any of the movement sensor, the pressure sensor, the methane sensor, the thermometer, or the humidity sensor.

13. The bandage assembly of claim 9, wherein the radio transceiver generates a wake up signal when the radio transceiver detects RF energy generated by said mobile communications device.

14. The bandage assembly of claim 13, wherein the wake up signal is transmitted to the microcontroller and any of the movement sensor, pressure sensor, methane sensor, thermometer or humidity sensor.

15. The bandage assembly of claim 9, wherein said radio transceiver retrieves data stored in said memory by using said harvested radio frequency energy while the microcontroller and any of the movement sensor, pressure sensor, methane sensor, thermometer or humidity sensor remain in a sleep state or power off mode.

16. The bandage assembly of claim 2, wherein the microcontroller controls transfer of data from any of the movement sensor, pressure sensor, methane sensor, thermometer or humidity sensor and the memory.

17. The bandage assembly of claim 2, wherein the memory, the microcontroller, the radio transceiver, and any of the movement sensor, pressure sensor, methane sensor, thermometer or humidity sensor are embedded within the bandage member.

18. The bandage assembly of claim 1, further comprising at least one of a pH sensor, oxygen sensor, uric acid sensor, or a capacitive sensor.
